(19) United States
(12) Patent Application Publication
(10) Pub. No.: US 2013/0210099 A1
(43) Pub. Date: Aug. 15, 2013

(54) METHOD FOR THE PRODUCTION OF ERYTHRITOL

(71) Applicants: Astrid Mach-Aigner, Untertullnerbach (AT); Robert Mach, Untertullnerbach (AT)

(72) Inventors: Astrid Mach-Aigner, Untertullnerbach (AT); Robert Mach, Untertullnerbach (AT)

(73) Assignee: ANNIKKI GMBH, A-Graz (AT)

(21) Appl. No.: 13/876,722

(22) PCT Filed: Sep. 28, 2011
(86) PCT No.: PCT/EP2011/066837

§ 371 Date: Apr. 25, 2013

(30) Foreign Application Priority Data
Sep. 30, 2010 (EP) 10183799.5

(51) Int. Cl. C12P 7/18

(52) U.S. Cl. CPC C12P 7/18
USPC 435/158

(57) ABSTRACT

The invention relates to a method for the production of erythritol, in which erythrose is reduced to erythritol using a NADPH-specific enzyme, wherein the enzyme is an erythrose reductase which was isolated from the group of saprophytes selected from the strains of Hypocrea, Gibberella, Aspergillus and Penicillium. Hereby it is possible to produce erythritol in high yields.

The invention relates to a method for the production of erythritol, wherein erythrose is reduced to erythritol using a NADPH-specific enzyme.

Polyols are carbohydrates whose carbonyl group (aldehyde or ketone) has been reduced to a primary or secondary hydroxyl group. These compounds are used in a variety of applications to provide functional foods and nutraceuticals with value-added properties. Typical commercially available polyols include sorbitol, mannitol, xylitol, lactitol, maltitol, and erythritol.

Erythritol is a naturally occurring substance that is widely distributed in nature. It occurs as a metabolite or storage compound in seaweeds and fungi and is also a component of a number of common fruits, such as melons, grapes and pears. It occurs frequently in fermented foods (soy sauce), drinks (wines and beers), and in processed vegetables such as miso bean paste.

A 10% solution of erythritol has 60% to 70% of the sweetness of sucrose. Eythritol can be safely used as a noncariogenic sweetener in foods as it cannot be fermented by the bacteria that cause dental caries. Moreover, it is low in calories, it lacks the laxative properties that other sweeteners have and it is also suitable for diabetics. It has a very high negative heat capacity, providing a strong cooling effect when dissolved.

Erythritol can be synthesized, for example, from dialdehyde starch by a high-temperature chemical reaction in the presence of a nickel catalyst. This process has, however, not been industrialized because of its low efficiency. Large-scale production of erythritol uses fermentative processes with pure glucose, sucrose, and glucose from chemically and enzymatically hydrolyzed wheat and corn starches. Furthermore, it can be produced by microbial methods that utilize osmophilic yeasts and some bacteria (Ryu, Y. W. et al., J. Ind. Microbiol. Biotechnol. 25: 100-103, 2000; Takafumi Kasumi, JARQ 29, 49-55, 1995).

The erythritol found in yeast and fungus species is produced via the so called pentose phosphate pathway. Hereby, erythritol is synthesized from D-erythrose-4-phosphate through dephosphorylation and subsequent reduction of erythrose. In the final step of this pathway erythrose reductase (ER) is playing a key role as catalyst.

Several erythrose reductases have been identified and characterized to date, e.g., erythrose reductase from Candida magnoliae (Lee et al., Appl. Environ. Microbiol. 69: 3710-3718, 2003) and from Torula corallina (Lee et al., Appl. Environ. Microbiol. 68: 4534-4538, 2002).

So far, research efforts have focused on the discovery and selection of microorganisms capable of synthesizing erythritol in high yields, as well as on the optimization of fermentation processes (Moon et al., Appl. Microbiol. Biotechnol. 86: 1017-1025, 2010). The U.S. Pat. No. 6,287,830 discloses a mutant strain of Candida magnoliae, which provides high yields of erythritol under suitable conditions of culture.

The invention has as its object to provide a method which enables production of erythritol in high yields. Furthermore, it should enable the cost-efficient use of renewable biomass as the source for microbial production.

This object is achieved with a method of the initially mentioned kind, wherein the enzyme is an erythrose reductase which was isolated from the group of saprophytes selected from the strains of Hypocrea, Gibberella, Aspergillus and Penicillium.

It has been found that the said saprophytic fungal strains code for an enzyme which has erythrose reductase activity and, thus, reduces erythrose to erythritol in the presence of NADPH.

In the context of the present invention, the term “erythrose reductase” is defined as an enzyme that catalyzes reversibly the oxidation of an aldose to an alditol, wherein the specific activity for reduction of erythrose is predominant. The erythrose reductase of the present invention accordingly exhibits very high specificity and affinity for the substrate erythrose. In the prior art, several enzymes have been described to have a certain degree of catalytic activity for the reduction of erythritol. All those enzymes were, however, shown to exhibit higher specific activity for the reduction of substrates other than erythrose, for example, glycerol, and therefore do not qualify as erythrose reductases according to the above definition.

The present invention further requires prior isolation of the enzyme from the saprophytic strain and its characterization as an erythrose reductase.

Preferably, the saprophyte is selected from the group of strains consisting of Hypocrea jecorina (Trichoderma reesei), Gibberella zeae, Aspergillus niger, Aspergillus nidulans, Aspergillus oryzae and Penicillium chrysogenum.

According to a preferred embodiment of the invention, the saprophyte from which the erythrose reductase was isolated is Trichoderma reesei. In particular, the erythrose reductase is encoded by nucleic acid sequence SEQ ID NO:1. It is also preferred that the erythrose reductase comprises the amino acid sequence SEQ ID NO:2.

A further preferred embodiment is characterized in that the saprophyte from which the erythrose reductase was isolated is Aspergillus niger or Aspergillus nidulans.

The sequence identities of the proteins with erythrose reductase activity produced by the other saprophytes used according to the present invention are 69% for Gibberella zeae, 65% for Aspergillus niger, 66% for Aspergillus nidulans, 62% for Aspergillus oryzae, and 62% for Penicillium chrysogenum, based on the protein from Trichoderma reesei (SEQ ID NO:2).

It is particularly preferred that the erythrose reductase used for the production of erythritol is overexpressed by a recombinant strain.

A preferred embodiment of the invention is characterized in that the method for the production of erythritol is carried out as a whole cell process.

The saprophytes which are used according to the present invention advantageously differ from other microorganisms in that erythritol has not to be produced (indirectly) by intensive glucose feeding (40%) but in that the required sugar compound which is needed as the energy source (D-xylose in the present case) is provided via the saprophytic activity of the fungi. As is generally known, saprophytes are organisms that do not produce their nutrients by photosynthesis but derive them from decaying organic matter. Favourably, lignocellulosic biomass such as straw can thus be utilized because the saprophytes are provided with a native degradation pathway in regard to such material, resulting in xylose which continues into the pentose phosphate pathway leading to erythrose. In this way, it is feasible to produce erythritol from low-cost renewable biomass.

A further preferred embodiment of the invention is characterized in that overexpression of the erythrose reductase-coding gene of the recombinant strain is controlled by a promotor which is induced by xylose. Hereby, the production of erythritol can be increased in a simple manner.

The invention is illustrated and explained in more detail by way of the following examples.

### EXAMPLES

### Microbial Strains And Growth Conditions

E. coli BL21 (DE3) pLysS and JM109 (both from Promega, Wis., US) were used as recipient strains for plasmid transformation and maintained on LB/amp media containing 1% (w/v) peptone, 1% (w/v) NaC1, 0.5% (w/v) yeast extract and ampicillin (100 m/mL).

### PCR of the err1 Structural Gene

An iCycler (Bio-Rad, Herkules, US) was used to run 30 cycles of 20 s 95° C., 20 s 59° C. and 1 min 72° C. We applied a 50 μL reaction comprising 5× Flexi buffer (Promega), 2.5 mM MgCl, 6.25 μM primer forward and reverse, 0.2 mM dNTPs, 1 U GoTaq Polymerase (Promega) and H. jecorina cDNA as a template. Primers that have been used are err1_SalITf (5′-ATAGTCGACTATGTCTTCCGGAAGGACC-3′) and err1_NotIr (5′-TATGCGGCCGCTTACAGCTTGATGACAGCAGTG-3′), introducing a SalI and a NotI restriction enzyme site at the 5′- or 3′-end of the amplicon, respectively. The obtained 996 bp-fragment was subcloned into the pJET/1.2 vector (Fermentas, St. Leon-Rot, Germany) using the CloneJET PCR Cloning Kit (Fermentas) and E. coli JM109 thermo-competent cells (Promega) according to the manufacturers instructions to create pARMA2T. Via the introduced restriction enzyme sites SalI and NotI err1 was released from pARMA2T and further cloned into the expression vector pGEX-4T-2 (GE Healthcare) to create pARMA2. The insertion of the err1 structural gene without mutations was confirmed by sequencing of the vector insert (MWG, Ebersberg, Germany).

### Transformation of E. coli

The transformation of pARMA2T or pARMA2 in E. coli JM109 or BL21 (DE3) pLysS cells, respectively, was carried out according to a standard protocol from Promega). Therefore, 10 μl-ligation mixture or 0.1 μg (1 μl) of the pARMA2 plasmid were provided in one reaction tube and 100 μl of E. coli JM109 or BL21 (DE3) pLysS competent cells, respectively, were mixed and put on ice for 20 min. The uptake of plasmid-DNA was initiated by heat shock. Therefor, the tube was placed on the thermo block at 42° C. for 1.15-1.30 min. Then the tube was quickly put on ice for 2 min and 900 μl SOC medium (2% (w/v) peptones, 0.5% (w/v) yeast extract, 1M NaCl, M KCl, 1M MgCl2 or MgSO4 and 2M glucose) was added. For regeneration, the tube was placed on the thermo block at 37° C. for 1 h with gentle shaking. Thereafter, cells were harvested by centrifugation (5000 g, 2 min) and plated on LB/amp medium.

### Isolation of Plasmid-DNA Out of Bacterial Colonies

Selected E. coli colonies, putatively containing pARMA2T or pARMA2, were cultivated in 4 mL LB/amp media at 37° C. over night. Cells were harvested by centrifugation at room temperature and plasmid-DNA preparation was performed by precipitating genomic DNA, cell debris and proteins as follows: After the cell pellet of each colony was properly mixed with 200 μl Buffer P1 (QIAGEN, Valencia, Calif.), 200 μl Buffer P2 (QIAGEN) were added, mixed thoroughly by inverting the sealed tube 4-6 times. Then the tubes were incubated at room temperature for 5 minutes. After incubating 200 μl Buffer P3 (QIAGEN) were added, inverted carefully 4-6 times and incubated on ice for 5 minutes. After 5 minutes the tubes got centrifuged at 13000×g for 10 minutes. The supernatant was transferred properly in a new tube and 450 μl isopropanol were added, inverted 4-6 times, incubated on ice for 10 minutes and centrifuged at 13000 x g for 30 minutes at 4° C. The supernatant was removed and 1 mL 70% ethanol was added to the plasmid-DNA pellet and mixed properly. The last centrifugation step was performed at 13000×g for 15 minutes at 4° C. The supernatant was removed and the pellet was dried on a thermo block at 50° C. Afterwards the white pellet was dissolved in 50 μl distilled water and put on a thermo block at 65° C. for better solving. The purified plasmid-DNA of each colony was analyzed by agarose gel electrophoresis. Plasmid-DNA of one colony, which showed intense bands, was purified via QIAGEN PCR Purification Kit, following the manufacturer's instructions and again analyzed by agarose gel electrophoresis.

### Preparation of Large-Scale Cultures And Induction of Protein Expression

A single colony of E. coli BL21 cells containing pARMA2 for GST::err1 expression was used for inoculating 4 mL LB/amp media and got incubated for 8 h at 37° C. with shaking at 160 rpm. The pre-culture was diluted 1:10 into 50 mL LB/amp media and grown at 37° C. with shaking at 160 rpm until it reached an OD600 of 1.676. The 50-mL-culture was diluted to an OD600 of 0.2 in a 250-mL-culture. Incubation at 37° C. with shaking was continued until the culture reached an OD600 of 0.752. At this point, the OD600 was optimal for induction which was initiated by adding IPTG (20 mg/mL) to a final concentration of 0.1 mM, and incubation was continued for additional 3 hours. The 250-mL-culture was transferred to a 50 mL tube and centrifuged at 4000 rpm for 5 minutes at 4° C. The supernatant was discarded, the cell pellet was solved in 2 mL PBS/Triton X-100 buffer and transferred to a 15 mL tube and placed on ice.

### Extraction of the GST-err1 Fusion Proteins

The cells were disrupted on ice with a sonicator in short bursts with following experimental settings: Cycle 50, 70% energy, 30 seconds sonication and 30 seconds pause, repeated 4 times. Bacterial lysates were removed by centrifugation at 5000 rpm for 10 min and 0.5 mL of the clear supernatant (cell-free extract) were transferred to a reaction tube and stored at 80° C. after adding 20% (v/v) glycerol.

### Purification of the GST-err1 Fusion Proteins

The GST fusion proteins in the cell-free extract were purified by affinity chromatography using glutathione immobilized to a matrix. Therefore, glutathione/sepharose columns (Amersham Bioscience, part of GE Healthcare, Conn., US) with 1 mL column volume were used for purification and the following buffers were prepared:
Binding buffer: PBS buffer, containing 140 mM NaCl, 2.6 mM KCl, 10 mM Na2HPO4, 1.8 mM KH2PO4, pH 7.3 and adding protease inhibitors 1 μM pepstatin A, 1 μM leupeptin, 0.5 μM PMSF right before usage.
Elution buffer: 50 mM Tris-HCl, 10 mM glutathione, pH 8.0, adding protease inhibitors 1 μM pepstatin A, 1 μM leupeptin, 0.5 μM PMSF right before elution.

Before applying to the column, the cell-free extract was filtered through a 0.45 μm filter and the column was equilibrated by 10-fold column volumes of binding buffer and a flow rate of 1 mL/min was maintained. Then, cell-free extract was applied on the column with a flow rate of 0.5 mL/min, the eluate was collected and stored at 4° C. for analysis and the column was washed with 10-fold column volumes of binding buffer. The GST fusion proteins were eluted with 5-fold column volumes of elution buffer and collected as 0.5 mL aliquots in 10 1.5mL reaction tubes.

The concentration of each collected fraction was measured via NanoDrop at 280 nm and the fractions which showed the highest protein concentrations were united and divided equally to 4 reaction tubes. The aliquots were mixed with 20% (v/v) glycerol and stored at −80° C. for analysis.

### Verification of Purified Expression Products Via SDS-PAGE

The eluted expression products were verified and separated according to their size by SDS-PAGE at constant current of 12 mA (using the Mini-PROTEAN System, Bio-Rad) followed by PageBlue™ (Fermentas) protein staining according to a fast staining protocol for mini-gels according to the manufacturers instructions. Therefor, the following reagents were required: 5× Tris-glycine buffer containing SDS (pH 8.8), SDS-polyacrylamide gel containing 4-15% Tris-HCl (BioRad, cat. No. 161-1104), and 5×SDS loading dye, PageRuler™ Prestained Protein Ladder (10-170 kDa) (Fermentas), and PageBlue™ Protein Staining Solution (Fermentas) containing Coomassie Brilliant Blue G 250. On the gel, 45 μg (15 μl) purified GST-err1 fusion protein and 10 μl protein ladder were applied. After staining, the gel was stored in distilled water at 4° C. for later reference.

### Enzyme Assay Measuring NADPH-Decrease

### Enzyme Aassay

The following 1 mL assay was performed: 400 μM NADPH (in 10 mM sodium phosphate buffer, pH 7), and 25 μL purified GST::err1 enzyme were mixed in an lmL quartz cuvette and incubated at 30° C. for 1 min. Then adding D-erythrose to a final concentration of 10 mM started the reaction. The decrease of NADPH concentration was measured in a spectrophotometer at 340 nm. As a control, a blank was performed using water instead of D-erythrose. As a reference this assay was repeated using glyoxal, Larabinose, L-arabitol, D-xylose, xylitol, D-xylulose, and L-xylulose as substrates.

### HPLC Analysis

### Enzyme Assay

The following 1 mL assays have been performed prior to HPLC analysis: Assay 1 comprised 4 mM NADPH (in 10 mM sodium phosphate buffer, pH 7), 25 μL purified GST::err1 enzyme, 5 mM erythrose and was incubated at 25° C. for 2 hours. Assay 2 was performed according to assay 1, but was incubated at 37° C. for 2 h. Both assays were stopped by incubating at 85° C. for 10 min and afterwards were filtered using 0.2 μm filters. As negative control assay 3 was performed like assay 1, but was immediately stopped by incubation at 85° C. for 10 min and assay 4 was performed according to assay 1, but sodium phosphate buffer instead of erythrose was used and it was immediately stopped at 85° C. for 10 min.

### HPLC Analysis

Analyses were performed using a Thermo Finnigan Surveyor HPLC instrument (Thermo Fisher Scientific, MA, US). All 10 μL samples were injected onto a Rezex RHM-Monosaccharide column (H+, 8%, 150×7.8 mm; Phenomenex, CA, US). Water was used as the mobile phase and elution was followed at 85° C., applying a flow rate of 0.3 mL/min for 20 min. 1 mM D-Erythrose (Sigma) and erythritol (Sigma) were used as reference substances.

### Overexpression of err1

The native T. reesei pki (pyruvate kinase-encoding) and bxl1 (beta-xylosidase-encoding, this enzyme cleaves the linkage between the two D-xylose units of which xylobiose consists of) promoters were used for overexpression of errl. This leads to a constitutive (pki) or an D-xylose inducible (bxl1) expression of errl. In both cases the cbh2 (cellobiohydrolase II-encoding) terminator was applied.

The resulting recombinant T. reesei strain which converts D-xylose via erythrose to erythritol can be fed with D-xylose or can grow on hemicellulosic substrates and provide D-xylose by its own saprophytic activity.

The D-xylose inducible system has benefits because D-xylose is an indispensable substance (educt or metabolite) in this conversion anyway. Sometimes in nature, problems occur with systems which are self-inducible, e.g., because the organism's cell integrity becomes disturbed. In such a case, a constitutive system can prove advantageous.

Apart from the above promotors, other constitutive, e.g., tef, gpd, or inducible promoters, e.g., cbh1, xyn1 can be applied as well.

### Results

The GLD1 protein from Trichoderma reesei, initially described as glycerol-dehydrogenase (GLD) by Liepins et al. (FEBS Journal 273 (2006), 4429-4235), has been found to exhibit erythrose reductase activity contrary to the earlier assumption, because it has the capability to convert erythrose to erythritol. The tests that show this finding are described in the following. Therefore, this enzyme was termed Erythrose reductase 1 (encoded by err1) by the inventors. Err1 was cloned and was subsequently used for production of erythritol in a recombinant T. reesei strain.

a) Determination of the enzymatic activity by analysis of the NADPH-decrease in an enzyme assay using GST::err1 fusion proteins with different substrates

According to Liepins et al. (2006) D-xylose was used as “negative” control and glyoxal as “positive” control because enzymatic activities on these substrates have been described.

| Substrate | Absorbance decrease (mAbs/min) |
|---|---|
| Glyoxal | −83.28 |
| D-xylose | −10.20 |
| Erythrose | −83.77 |

b) HPLC-Analyses of the enzymatic conversion of erythrose using heterologously expressed GST::err1 fusion proteins gave clear indication that erythrose is reduced to erythritol.

c) The determination of Vₘₐₓ and Kₘ according to Liepins et al. (2006) gave clear indication that the preferred substrate for Err1 is erythrose and the reduction of it is not only the result of any star activity. Lipiens et al. referred to D-glyceraldehyde and methylglyoxal as substrates with good activity for GLD1, and they could detect no activity using glycerol (ND, no detection). The tests carried out by the inventors showed that erythrose has approximately 10-fold higher activity (see Table 1).

TABLE 1

| Substrate | Vₘₐₓ (nkat/mg) | Kₘ (mM) | Vₘₐₓ/Kₘ (/s M) |
|---|---|---|---|
| D-Glyceraldehyde | 150 | 0.9 | 6100 |
| Glyoxal | 375 | 2.4 | 5800 |
| Glycerol | ND | ND | |
| D-Xylose | 450 | 334 | 48 |
| Erythrose | 530 | 0.016 | 33125 |

1. A method for the production of erythritol, wherein erythrose is reduced to erythritol using a NADPH-specific enzyme, wherein the NADPH-specific enzyme is an erythrose reductase which was isolated from the group of saprophytes selected from the strains of Hypocrea, Gibberella, Aspergillus and Penicillium.

2. The method according to claim 1, characterized in that wherein the saprophyte is selected from the group of strains consisting of Hypocrea jecorina (Trichoderma reesei), Gibberella zeae, Aspergillus niger, Aspergillus nidulans, Aspergillus oryzae and Penicillium chrysogenum.

3. The method according to claim 1, wherein the saprophyte is Trichoderma reesei.

4. The method according to claim 3, wherein the erythrose reductase is encoded by nucleic acid sequence SEQ ID NO: 1.

5. The method according to claim 3, wherein the erythrose reductase comprises the amino acid sequence SEQ ID NO:2.

6. The method according to claim 1, wherein the saprophyte is Aspergillus niger or Aspergillus nidulans.

7. The method according to any of claim 1, wherein the erythrose reductase used for the production of erythritol is overexpressed by a recombinant strain.

8. The method according to claim 7, carried out as a whole cell process.

9. The method according to claim 7, wherein overexpression of the erythrose reductase-coding gene of the recombinant strain is controlled by a promotor which is induced by xylose.


SEQUENCE LISTING (4 sequences)

SEQ ID NO: 1 - DNA, 996 bp, Hypocrea jecorina
atgtcttccggaaggaccgtcaccctcaacaccggctacaagatcccccagatcggctacggcacctggcaggccgctcccggcgaggtcggcgctggtgtctttgaggccctcaaggttggctaccgccacctcgacctggccaaggtctacggcaaccagaaggaggttggtgagggcatcaagaaggctcttgctgaggtccccggcctgaagcgcgaggatattttcatcacctccaagctgtggaacaactcccacaagcccgaggacgtcgagcccgctctcgacgacaccctggccgagcttggcctcgactaccttgacctctacctcatccactggcccgttgcctttgctcccggcgccgacctcttccccaagtccgaggacggctccgaggtgcagctcaaccagaatgtgtccattgtccagacctggaaggccatgaccgagctgcccaagtccaaggtccgctccgtcggtgtctccaactttaccattgagcacctcgacgccgtcatcgaggccaccggcgtcgtccccgccgtcaaccagatcgagcgccacccccgcctccccaaccagcccctgatcgactactgcgccaagaagggcatcatcatcaccgcctactccgcctttggcaacaacacaaagggcctgcccctgctcgtcagctccgacgaggtcaaggccgtcgccgacaacctgtccaagaagcagggcaagaccgtcactcccgcccaggtcatcctcgcctggtcccagattggtggccacaccgtcattcccaagtccgtcaccaaggcgcgcattgcggagaacttccaggaggttgagctggatgacgaggccattgctgcgctgaacaagttgggcgagaagcctcagcggttcaacattccttacacctacaagcctaggtggaacattaacctgttcaacaccgaggaggagaaggccgctgcccacactgctgtcatcaagctgtaa

SEQ ID NO: 2 - protein, 331 aa, Hypocrea jecorina
MSSGRTVTLNTGYKIPQIGYGTWQAAPGEVGAGVFEALKVGYRHLDLAKVYGNQKEVGEGIKKALAEVPGLKREDIFITSKLWNNSHKPEDVEPALDDTLAELGLDYLDLYLIHWPVAFAPGADLFPKSEDGSEVQLNQNVSIVQTWKAMTELPKSKVRSVGVSNFTIEHLDAVIEATGVVPAVNQIERHPRLPNQPLIDYCAKKGIIITAYSAFGNNTKGLPLLVSSDEVKAVADNLSKKQGKTVTPAQVILAWSQIGGHTVIPKSVTKARIAENFQEVELDDEAIAALNKLGEKPQRFNIPYTYKPRWNINLFNTEEEKAAAHTAVIKL

SEQ ID NO: 3 - DNA, 28 bp, Artificial
  PCR primer err1_SalITf
atagtcgactatgtcttccggaaggacc

SEQ ID NO: 4 - DNA, 33 bp, Artificial
  PCR primer err1_NotIr
tatgcggccgcttacagcttgatgacagcagtg